(19) United States
(12) Patent Application Publication
(10) Pub. No.: US 2016/0220101 A1
(43) Pub. Date: Aug. 4, 2016

(54) DEVICE

(71) Applicant: OLYMPUS CORPORATION, Tokyo (JP)

(72) Inventor: Shoei TSURUTA, Tokyo (JP)

(73) Assignee: OLYMPUS CORPORATION, Tokyo (JP)

(21) Appl. No.: 14/808,067

(22) Filed: Jul. 24, 2015

Related U.S. Application Data
(63) Continuation of application No. PCT/JP2014/084651, filed on Dec. 26, 2014.

(30) Foreign Application Priority Data
Nov. 11, 2013 (JP) 2013-233253

(51) Int. Cl. A61B 1/018; A61B 17/34; A61B 17/02

(52) U.S. Cl. CPC A61B 1/018; A61B 17/0218; A61B 17/3423; A61B 2017/00411

(57) ABSTRACT

Provided is a treatment tool configured so that no induction current flows through a manipulation wire of a manipulation part manipulated by an operator. The treatment tool includes: the manipulation part that is manipulated by an operator; an elongated insertion portion that is elongated from the manipulation part and is inserted into a body of a subject; and the manipulation wire that passes through the elongated insertion portion to transmit manipulation at the manipulation part to a treatment portion on a forward-end side (distal-end) of the insertion part. The manipulation wire includes a first wire having one end connected to the treatment portion, to which AC magnetic field or AC electrical field is applied, and a second wire having one end connected to the manipulation part and the other end that is connected to the first wire but is insulated electrically from the first wire.

### TECHNICAL FIELD

The present invention relates to a medical device to receive or transmit power wirelessly.

### BACKGROUND ART

A trocar is combined with an inner needle having a sharp puncture needle at the forward end, and the inner needle in such a state is punctured through a body wall of a patient so as to be inserted into the abdominal cavity. After being inserted into the abdominal cavity, the inner needle is removed so as to leave the trocar at the body wall, and then the trocar is used as a guide tube for a treatment tool that is for treatment in the abdominal cavity.

Some treatment tools inserted into a body of the subject via an insertion hole of a trocar are connected to a cable to receive power required for the treatment. Such a cable hinders the manipulation by an operator during operation and degrades the operability.

In the case of a treatment tool including a manipulation wire to transmit the manipulation by an operator mechanically, AC magnetic field generated at the power-transmission coil is applied to the manipulation wire as well, meaning that high-frequency induction current may flow through the manipulation wire. The manipulation wire is inserted in the manipulation portion manipulated by the operator, and so the high-frequency current may flow through the manipulation portion internally. The induction current flowing through the manipulation wire may adversely affect the power transmission/reception efficiency.

In the case of a flexible endoscope equipped with a power-transmission coil as well, when AC magnetic field is generated to transmit power to a treatment tool inserted into a channel wirelessly, induction current may flow through a manipulation wire of a manipulation portion with which an operator manipulates a curved part.

A medical device is desired, which is configured so that little or no induction current flows through a manipulation wire of a manipulation portion manipulated by an operator.

### SUMMARY OF THE INVENTION

An embodiment of the present invention aims to provide a medical device which is configured so that little or no induction current flows through a manipulation wire of a manipulation portion manipulated by an operator.

### BRIEF DESCRIPTION OF THE DRAWINGS

FIG. 1 is a partial cross-sectional view of a treatment tool of a first embodiment.

FIG. 2 is an enlarged partial cross-sectional view of a treatment tool of the first embodiment.

FIG. 3 is a cross-sectional view of a manipulation wire of the treatment tool in the first embodiment.

FIG. 4 is an exploded cross-sectional view of the treatment tool that is a modification example 1 of the first embodiment.

FIG. 5A is a cross-sectional view of a treatment tool that is modification example 2 of the first embodiment.

FIG. 5B is a cross-sectional view taken along the line VB-VB of FIG. 5A that is modification example 2 of the first embodiment.

FIG. 6 is a cross-sectional view of a treatment tool that is modification example 3 of the first embodiment.

FIG. 7A is a schematic representation of a manipulation wire of a treatment tool that is modification example 4 of the first embodiment.

FIG. 7B is a schematic representation of a manipulation wire of a treatment tool that is modification example 5 of the first embodiment.

FIG. 7C is a schematic representation of a manipulation wire of a treatment tool that is modification example 6 of the first embodiment.

FIG. 7D is a schematic representation of a manipulation wire of a treatment tool that is modification example 7 of the first embodiment.

FIG. 8 is an illustration of a treatment tool of a second embodiment and an endoscope of a third embodiment.

FIG. 9 is a partial cross-sectional view of the treatment tool and endoscope of the second embodiment.

FIG. 10 is a partial cross-sectional view of the endoscope of the third embodiment.

### DETAILED DESCRIPTION OF THE INVENTION

As used herein, the term “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

As used herein, the term “substantially” refers to the complete or nearly complete extent or degree of an action, characteristic, property, state, structure, item, or result. For example, an object that is “substantially” parallel would mean that the object is either completely parallel or nearly completely parallel. The exact allowable degree of deviation from absolute completeness may in some cases depend on the specific context. However, generally speaking the nearness of completion will be so as to have the same overall result as if absolute and total completion were obtained.

### First Embodiment

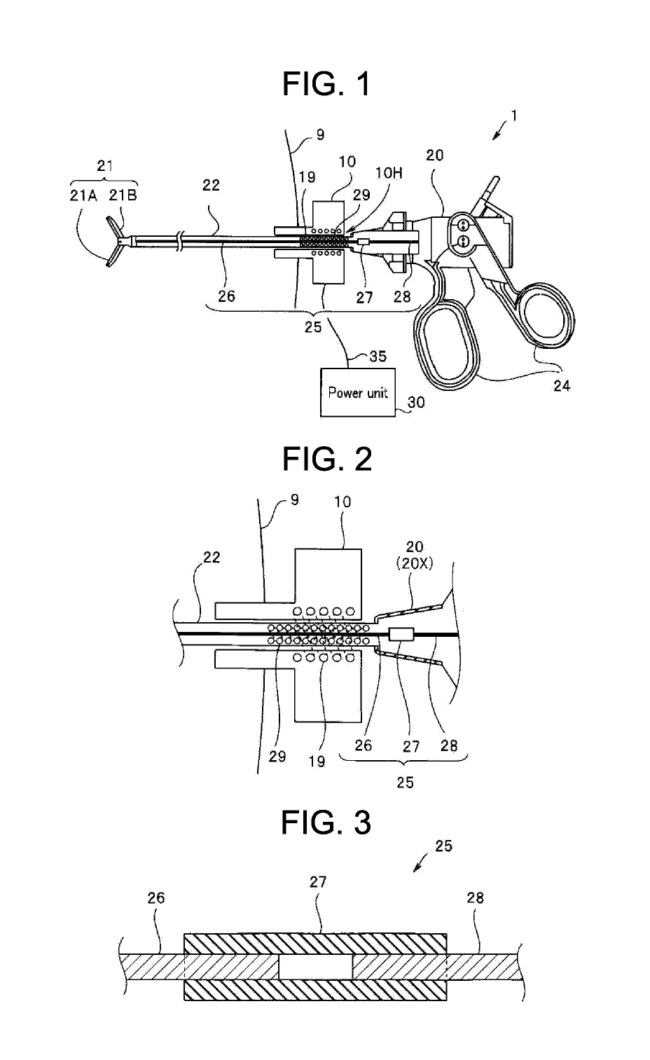

Referring to FIGS. 1 to 3, the following describes a treatment tool 20 that is a device of a first embodiment. As illustrated in FIG. 1, the treatment tool 20 makes up an operating system 1 with a trocar 10 as an insertion aid and a power unit 30. The treatment tool 20 for surgical operation can be inserted into a body of a subject 9 via an insertion hole 10H of the trocar 10 that is punctured through a body wall of the subject 9. Although the operating system 1 allows an endoscope or the like also to be inserted into the body via another trocar, the descriptions thereof are omitted.

The treatment tool 20 can be a high-frequency treatment tool that includes: a manipulation portion 24; an elongated insertion portion 22 that can be inserted into a body of the subject 9; an optional treatment portion 21 that is disposed on the forward end side (distal-end) of the elongated insertion portion 22; a manipulation wire 25 and a power-reception coil (power-reception portion) 29. The manipulation wire 25 can be made of metal with high rigidity, e.g., stainless steel (SUS) or nickel titanium (Ni—Ti) alloy, through which the elongated insertion portion 22 can be inserted, and is configured to transmit the manipulation at the manipulation portion 24 to the treatment portion 21.

The treatment portion 21 can be used for treatment in the body of the subject 9 and can include a pair of jaws 21A and 22B that can open and close. For example, when the operator grasps the manipulation portion 24, the jaws 21A and 22B are closed, and when the operator releases the manipulation portion 24, the jaws 21A and 22B are open.

While pinching tissue to be treated between the jaws 21A and 22B, high-frequency current can be applied to the jaws 21A and 22B for treatment, such as for incision or for stopping of bleeding.

Power for treatment can be transmitted wirelessly to the treatment tool 20 from the trocar 10.

For this transmission, a solenoid-type power-transmission coil 19 is wound around the insertion hole 10H of the trocar 10 at the outer periphery. When receiving AC power from a power unit 30 via a cable 35, the power-transmission coil 19 generates AC magnetic field.

The power unit 30 outputs high-frequency and power of between about 10 W to about 100 W, for example. The AC magnetic field generated by the power-transmission coil 19 has a frequency that can be selected appropriately in the range of about 100 kHz to about 20 MHz, for example, about 13.56 MHz.

Then the power-reception coil 29 of the treatment tool 20 is inductively coupled with the power-transmission coil 19 of the trocar 10, such that the power-reception coil 29 receives power wirelessly via the AC magnetic field. The power received by the power-reception coil 29 is converted into high-frequency power at a power-reception circuit (not illustrated), which is then applied to the jaws 21A and 22B.

In the treatment tool, the manipulation wire 25 is inserted through the power-reception coil 29 internally. Thus, AC magnetic field generated by the power-transmission coil 19 of the trocar 10 is applied to the manipulation wire 25 as well. AC magnetic field is applied to the manipulation wire 25 when the manipulation wire is disposed outside of the power-reception coil 29 also. Induction current therefore flows through the manipulation wire 25.

As illustrated in FIG. 2, the manipulation wire 25 of the treatment tool 20 includes a first wire 26 having one end connected to the treatment portion 21 on the forward end side of the elongated insertion portion 22, and a second wire 28 having one end connected to the manipulation portion 24. Then the first wire 26 and the second wire 28 are connected mechanically, but are substantially insulated electrically.

As illustrated in FIG. 3, for example, the first wire 26 and the second wire 28 are both made of metal, and are connected with a heat-shrinkable tube 27 so that they are not in direct contact with each other.

The heat-shrinkable tube 27 is a shape-memory plastic tube that shrinks into a pre-memorized shape by heating and is made of an insulating material, such as silicone resin or fluorine resin.

As illustrated in FIG. 2, the connecting part, i.e., the heat-shrinkable tube 27 is disposed closer to the manipulation portion 24 than the power-reception coil 29 when the power-transmission coil 19 and the power-reception coil 29 are electromagnetically coupled. Induction current flows through the first wire 26 due to the AC magnetic field generated by the power-transmission coil 19. The induction current, however, does not substantially flow to the second wire 28 from the first wire 26.

The treatment tool 20 is configured so that little or no induction current flows through the second wire 28 of the manipulation portion 24 manipulated by the operator. This can eliminate the risk of heat generated at the second wire 28 or a failure of the intensity of electromagnetic wave to achieve a specific absorption rate (SAR) due to induction current, for example.

At least one of the first wire 26B and the second wire 28 may be used as a signal line or a ground potential line.

High-frequency current may flow through an unexpected path. One example of a method to address this is illustrated in FIG. 2. The elongated insertion portion side of the treatment tool 20 can include one or more elements 20X made of a low dielectric loss insulating material. In other words, the treatment tool 20 and the manipulation portion 24 can be electrically insulated with an element 20X made of a relatively low dielectric loss insulating material, e.g., polystyrene, polyethylene, or fluorine resin having dielectric loss tangent of 0.01 or less. The element 20X may be a connecting member that connects removably with the manipulation portion 24 and the elongated insertion portion 22.

The above describes a bipolar-type high-frequency treatment tool as the treatment tool 20, which may be various treatment tools having a manipulation wire to manipulate a manipulation portion on the forward end side, e.g., a monopolar-type electrosurgical knife, and similar effects can be obtained therefrom.

The above describes the treatment tool 20 that receives power wirelessly via the power-reception coil 29 that is electromagnetically coupled with the power-transmission coil 19 of the trocar 10 as an insertion aid. The treatment tool may be one that receives power wirelessly via a power-reception electrode that can be capacitively-coupled with a power-transmission electrode (power-transmission portion) of an insertion aid, and such a configuration of the present invention has the same effects when induction current flows through the manipulation wire.

Thus, a medical device can include a manipulation wire that is made up of a first wire having one end connected to the forward end side, to which AC magnetic field or AC electrical field is applied, and a second wire having one end connected to the manipulation portion and the other end of the second wire being connected to the first wire and insulated from the first wire. In such a medical device, little or no induction current flows through the manipulation wire of the manipulation portion manipulated by the operator.

### Modification Example 1

The manipulation wire of the treatment tool 20 may include a first wire having one end connected to the treatment portion 21 on the forward end side and a second wire having one end connected to the manipulation portion 24 and the other end of the second wire connected to the first wire and being substantially electrically insulated from the first wire.

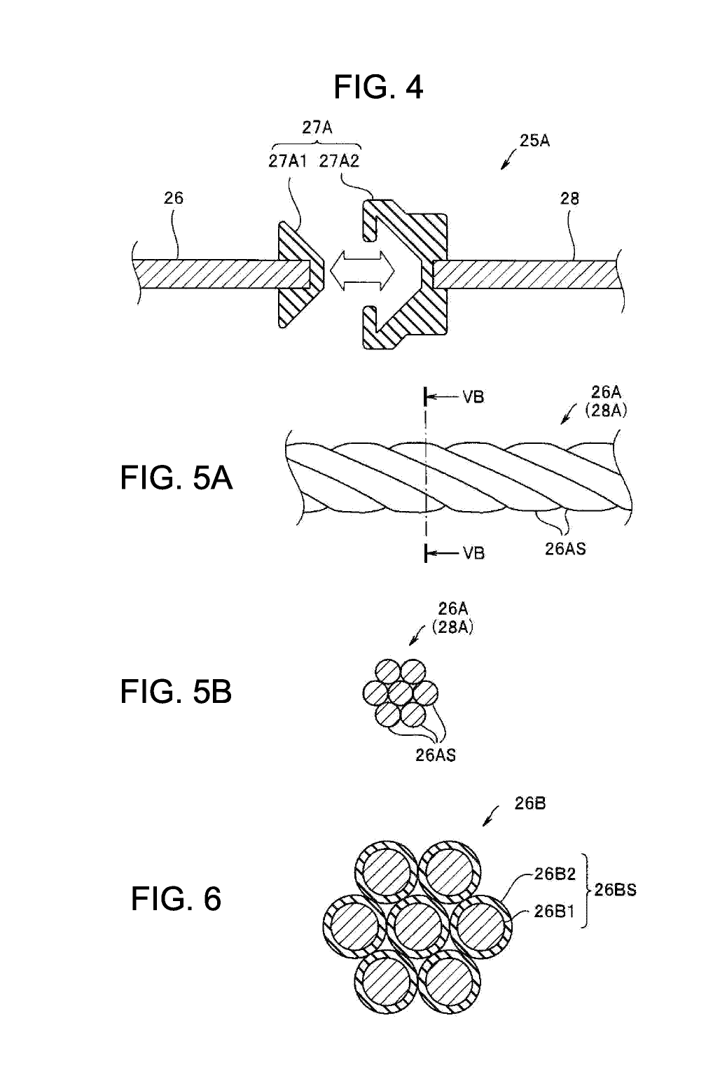

For example, a manipulation wire 25A of the treatment tool as modification example 1 in FIG. 4 includes a first wire 26 made of metal and a second wire 28 made of metal that are connected via a connecting member 27A including a first member 27A1 and a second member 27A2 that fit with each other. That is, the first wire 26 is joined with the first member 27A1, and the second wire 28 is joined with the second member 27A2.

The connecting member 27A may be an insulating material made of fluorine resin such as poly tetra fluoro ethylene (PTFE), or poly ether ether ketone (PEEK).

When the first wire 26 and the second wire 28 of the treatment tool are mechanically connected via a connecting member made of an insulating material, such a treatment tool has the substantially same effects as those of the treatment tool 20.

### Modification Example 2

As illustrated in FIG. 5A and FIG. 5B, any one of a first wire 26A and a second wire 28A of a treatment tool as modification example 2 is stranded wire including a plurality of metal element wires 26AS made of, for example, stainless steel (SUS) or nickel titanium (Ni—Ti) alloy, that are stranded into one.

Stranded wire is resistant to stretching and has good durability against bending.

### Modification Example 3

As illustrated in FIG. 6, in the manipulation wire of a treatment tool as modification example 3, at least a first wire 26B is stranded and includes metal element wires 26BS each including metal element wire 26B1 coated with an insulating material 26B2. The second wire also may have the same configuration as that of the first wire 26B.

AC magnetic field is applied to the first wire 26B. Since each metal element wire 26BS is insulated, meaning that eddy current generated has a shorter loop length, loss is relatively small and the amount of heat generated also is relatively small.

When the first wire 26B is used as a signal line, since each metal element wire 26B1 is relatively thin in diameter, increase in electrical resistance due to proximity effect can be suppressed. As a result, the first wire 26B can have good transmission efficiency and the amount of heat generated is relatively small.

Polyurethane can be used for coating of metal conducting wires. Coating of metal element wire used for a treatment tool can be made of a material having relatively high heat resistance, such as heat-resistant polyurethane, nylon, polyester, fluorine resin or polyparaxylylene.

### Modification Example 4

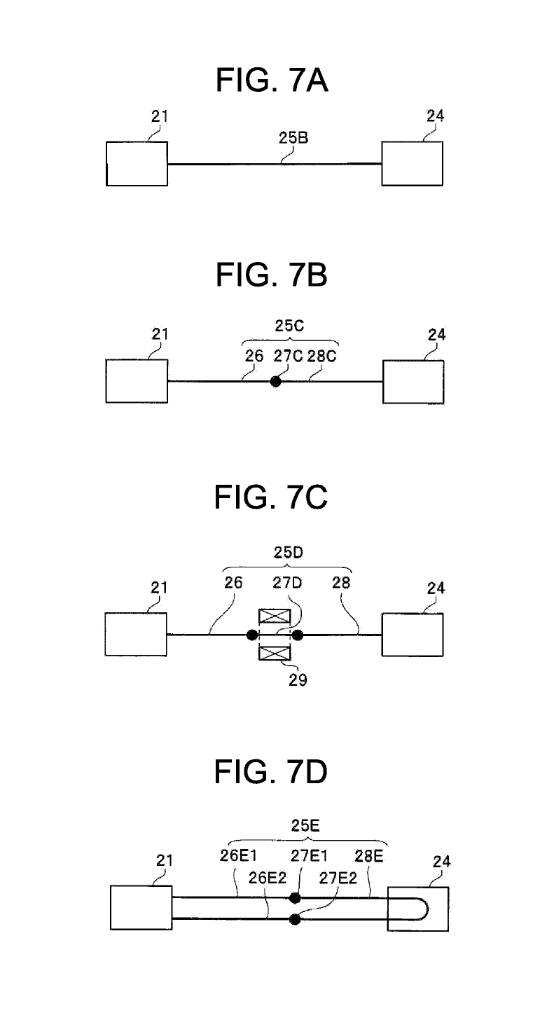

The manipulation wire can be made of metal or one manipulation wire 25B can be made of an insulating material illustrated in FIG. 7A in some specifications.

### Modification Example 5

As illustrated in FIG. 7B, a manipulation wire 25C may include a first wire 26 made of metal and a second wire 28C made of an insulating material that are connected via a connecting member 27C, and in this configuration also, little or no induction current flows through the second wire 28C of the manipulation portion 24 manipulated by an operator. The connecting member 27C may be an electrically-conductive member.

The insulating wire can be made of PEEK with relatively high rigidity. Stranded wire including a plurality of element wires made of an insulating material that are stranded may also be used.

### Modification Example 6

A manipulation wire 25D illustrated in FIG. 7C includes a connecting member that is an insulating wire 27D made of PEEK, for example. Since magnetic field can be applied to a part of the wire passing through the power-reception coil 29, i.e., a part passing through the power-transmission coil 19, relatively large induction current flows therethrough and eddy current is generated, which may degrade the power transmission/reception efficiency. To avoid or lessen this, insulating wire 27D can be used, which can prevent induction current from flowing into the manipulation portion side and can prevent the degradation of the power transmission/reception efficiency.

### Modification Example 7

A manipulation wire 25E illustrated in FIG. 7D includes two wires 26E1 and 26E2 connected to the treatment portion 21, and a wire 28E connected to the wires 26E1 and 26E2 via connecting members 27E1 and 27E2. The wire 28E is mechanically connected to the manipulation portion 24 not at an end portion but at portion near the middle. In the wire 28E, when the manipulation portion 24 rotates by wire 26E1 receiving tensile stress to the manipulation portion side, for example, the wire 26E2 receives compressive stress to the treatment part side.

Although the above describes the manipulation wire including a first wire and a second wire connected, three or more wires may be connected. In those embodiments, a connecting part made of an insulating material may be disposed at a portion closer to the manipulation portion than the position of the power-reception coil.

### Second Embodiment

A medical device of the present embodiment is a treatment tool 50 that is inserted into a channel of an endoscope 40. That is, an insertion aid for the treatment tool 50 is the endoscope 40 instead of the trocar 10.

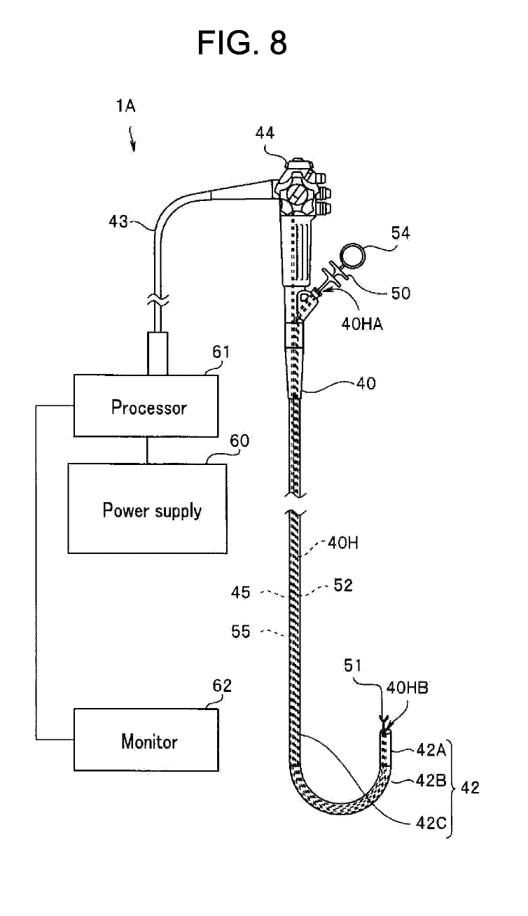

As illustrated in FIG. 8, an operating system 1A includes the treatment tool 50 of the present embodiment, the endoscope 40, a processor 61 that is connected to the endoscope 40 via a universal cord 43 and processes a signal, a power supply unit 60 and a monitor 62 to display an image.

The endoscope 40 includes a channel 40H that is an insertion hole into which an elongated insertion portion 42 to be inserted into a body of a subject is inserted. The channel 40H may be a flexible resin tube. The detailed configuration of the endoscope 40 will be described in a third embodiment.

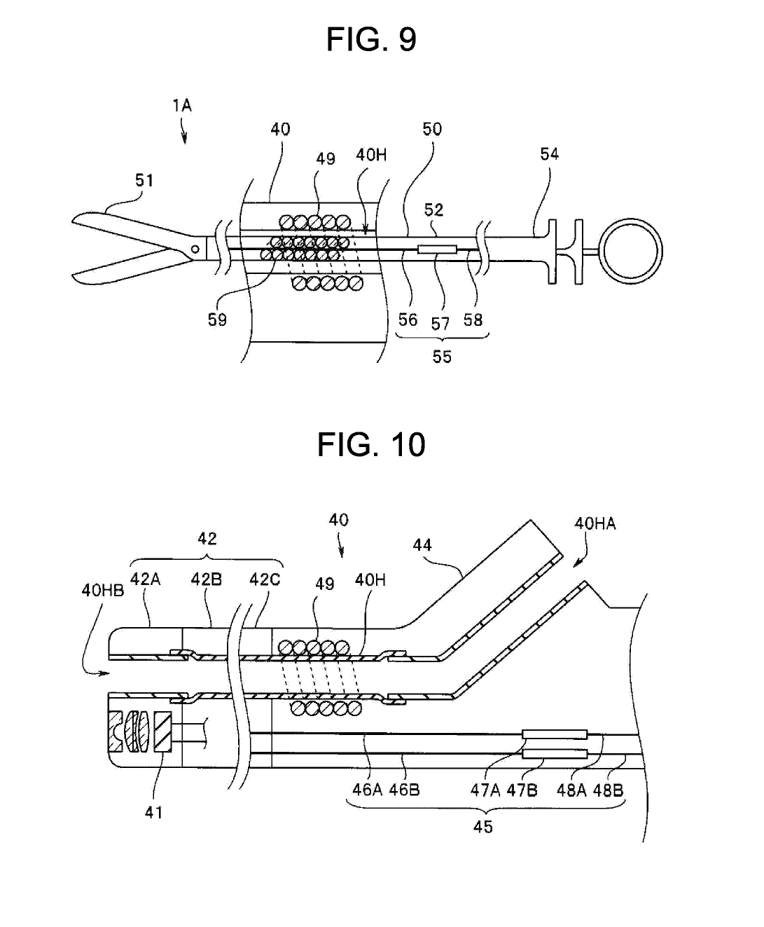

As illustrated in FIG. 9 and FIG. 10, the treatment tool 50 includes a manipulation portion 54 manipulated by an operator, a treatment part 51 on the forward end side, and an elongated insertion portion 52 into which a manipulation wire 55 is inserted, the manipulation wire transmitting the manipulation from the manipulation portion 54 to the treatment part 51. The treatment tool 50 is inserted from an insertion hole 40HA of the channel 40H in the manipulation portion 54 of the endoscope 40, and passes through the elongated insertion portion 42 (soft part 42C, curved part 42B, forward-end part 42A), and the treatment part 51 on the forward end side protrudes from an opening 40HB.

A power-transmission coil 49 wound around the channel 40H of the endoscope 40 generates AC magnetic field. Treatment is then performed at the treatment part 51 with power received by a power-reception coil 59 that is electromagnetically coupled with the power-transmission coil 49.

The manipulation wire 55 of the treatment tool 50 includes a first wire 56 having one end connected to the treatment part 51 on the forward end side of the elongated insertion portion 52, a second wire 58 having one end connected to the manipulation portion 54, and an insulating connecting member 57. The first wire 56 and the second wire 58 are connected mechanically, but are insulated electrically.

Although the insertion aid for the treatment tool 50 of the present embodiment is the endoscope 40, its basic configuration is similar as that of the treatment tool 20 of the first embodiment, and has a similar function. That is, little or no induction current flows through the manipulation wire 58 of the manipulation portion 54 manipulated by an operator.

The configurations of the modification examples of the first embodiment may be used in the treatment tool 50 of the present embodiment as well.

### Third Embodiment

A medical device of the present embodiment includes an endoscope 40 as an insertion aid. That is, an operating system 1A illustrated in FIG. 8 includes the treatment tool 50 of the second embodiment, and the endoscope 40 of the present embodiment.

The endoscope 40 as an insertion aid has a different basic configuration from that of the treatment tool 50 or the like. However, they are common in that AC magnetic field is applied to the manipulation wire.

As already described, the endoscope 40 includes a power-transmission coil 49 that generates AC magnetic field to transmit power to the treatment tool 50 inserted into the channel 40H. Then as illustrated in FIG. 8 and FIG. 10, the endoscope 40 includes a manipulation wire 45 that passes through the elongated insertion portion 42, the manipulation wire 45 being able to manipulate the curved part 42B that changes the direction of the forward-end part 42A at which an imaging part 41 is disposed.

As illustrated in FIG. 10, the manipulation wire 45 includes a first wire 46A and a second wire 48A that are connected via an insulating connecting member 47A, and a first wire 46B and a second wire 48B that are connected via an insulating connecting member 47B. One end of the first wire 46A and the other first wire 46B are connected to the curved part 42B on the forward end side. The other ends of the second wire 48A and the other second wire 48B are connected to an angle knob of the manipulation portion 54. The second wire 48A and the other second wire 48B may be one wire such that their other ends on the manipulation portion side are connected.

In the endoscope 40, AC magnetic field generated at the power-transmission coil 49 is applied to the manipulation wire 45 as well.

The first wires 46A, 46B and the second wires 48A, 48B, however, are connected mechanically, but are substantially insulated electrically.

Although the endoscope 40 of the present embodiment is an insertion aid, its basic configuration is similar as those of the treatment tools 20 and 50, and has a similar function. That is, little or no induction current flows through the manipulation wire 48A or 48B of the manipulation portion 44 manipulated by an operator.

The configurations of the modification examples of the first embodiment may be used in the endoscope 40 of the present embodiment as well.

The present invention is not limited to the above-described embodiments, and can be changed, combined and adapted variously without changing the gist of the present invention.

### DESCRIPTION OF REFERENCE NUMERALS

1, 1A operating system
10 trocar
19 power-transmission coil
20 treatment tool
21 treatment part
22 elongated insertion portion
24 manipulation portion
25 manipulation wire
26 first wire
27 heat-shrinkable tube
28 second wire
29 power-reception coil
30 power unit
40 endoscope
40H channel
42 elongated insertion portion
42A forward-end part
42B curved part
44 manipulation part
45 manipulation wire
49 power-transmission coil
50 treatment tool
51 treatment part
52 elongated insertion portion
54 manipulation part
55 manipulation wire
56 first wire
57 connecting member
58 second wire
59 power-reception coil
60 power unit

1. A device, the device comprising:
a manipulation portion;
an elongated insertion portion extending from the manipulation portion; and
a manipulation wire that extends from the manipulation portion, through the elongated insertion portion and is capable of transmitting a force from the manipulation portion to a distal-end side of the elongated insertion portion,
wherein the manipulation wire comprises a first wire and a second wire,
a first end of the first wire being extending to the distal-end side of the insertion portion, the first wire configured to receive an AC magnetic field or an AC electrical field, and
a first end of the second wire extending to the manipulation portion and a second end of the second wire operably connected to the first wire, wherein the second wire is substantially insulated electrically from the first wire.

2. The device according to claim 1,
wherein the first wire comprises one or more metals and the second wire comprises one or more metals are operably connected via a connecting member, the connecting member comprises of a material that is substantially insulating.

3. The device according to claim 2,
wherein the connecting member comprises one or more wires of a material that is substantially insulating.

4. The device according to claim 1,
wherein at least one of the first wire and the second wire comprise a stranded wire, and wherein the stranded wire comprises a plurality of stranded wires.

5. The device according to claim 4,
wherein the first wire is a stranded wire, and wherein the plurality of stranded wires are each coated with a substantially insulating material.

6. The device according to claim 1,
wherein the second wire is comprised of a substantially insulating material.

7. The device according to claim 1,
wherein the manipulation wire is comprised of a substantially insulating material.

8. The device according to claim 1, wherein the insertion portion further comprises: a power-reception portion, the power-reception portion configured to receive an alternating current AC magnetic field or configured to receive an AC electrical field.

9. The device according to claim 8,
wherein the insertion portion further comprises a treatment portion on the distal-end of the insertion portion, the treatment portion configured to receive the AC magnetic field or the AC electrical field from the power-reception portion.

10. A system, the system comprising:
a device, the device comprising:
a manipulation portion;
an elongated insertion portion extending from the manipulation portion; and
a manipulation wire that extends from the manipulation portion, through the elongated insertion portion and is capable of transmitting a force from the manipulation portion to a distal-end side of the elongated insertion portion,
wherein the manipulation wire comprises a first wire and a second wire,
a first end of the first wire being extending to the distal-end side of the insertion portion, the first wire configured to receive an AC magnetic field or an AC electrical field, and
a first end of the second wire extending to the manipulation portion and a second end of the second wire operably connected to the first wire, wherein the second wire is substantially insulated electrically from the first wire; and
an insertion aid, the insertion aid having an insertion hole formed therein, the insertion hole configured for the insertion portion to pass therethrough,
the insertion aid comprising a power-transmission portion configured to generate an alternating current (AC) magnetic field or an AC electrical field.

11. The system according to claim 10, wherein the insertion aid is a trocar.

12. The device according to claim 10, wherein the insertion aid is an endoscope, wherein the insertion hole is a channel in the endoscope.
